(19) United States
(12) Patent Application Publication
(10) Pub. No.: US 2025/0380896 A1
(43) Pub. Date: Dec. 18, 2025

(54) APPARATUS AND METHOD FOR EVALUATING CEREBRAL AUTOREGULATION

(71) Applicant: SEOUL NATIONAL UNIVERSITY HOSPITAL, Seoul (KR)

(72) Inventors: KEE WON KIM, Gyeonggi-do (KR); JUN MO KIM, Seoul (KR); HEE SOO KIM, Seoul (KR); SEUNG BO LEE, Seoul (KR)

(21) Appl. No.: 18/879,387

(22) PCT Filed: Apr. 14, 2023
(86) PCT No.: PCT/KR2023/005113

§ 371 Date: Dec. 27, 2024

(30) Foreign Application Priority Data
Jun. 30, 2022 (KR) 10-2022-0080432

(51) Int. Cl. A61B 5/00; A61B 5/0205; A61B 5/021; A61B 5/145

(52) U.S. Cl. CPC A61B 5/4064; A61B 5/0205; A61B 5/021; A61B 5/14542; A61B 5/7271; A61B 5/746; A61B 2505/05

(57) ABSTRACT

An apparatus for evaluating cerebral autoregulation includes a data acquisition unit configured to acquire blood pressure data and oxygen saturation data of a patient undergoing surgery, a correlation coefficient calculation unit configured to calculate correlation coefficients between the acquired blood pressure data and the acquired oxygen saturation data, a filtering unit configured to filter the calculated correlation coefficients using a moving average filter having a predetermined time window, and a cerebral autoregulation evaluation unit configured to evaluate the cerebral autoregulation of the patient undergoing surgery based on the filtered correlation coefficient.

### BACKGROUND

### 1. Technical Field

The present invention relates to a technique for evaluating cerebral autoregulation in real time during surgery.

### 2. Background Art

Cerebral autoregulation is a physiological mechanism which maintains cerebral blood flow at a constant level despite changes in a cerebral perfusion pressure. As long as the cerebral autoregulation is maintained intact, the brain can protect itself from excessively high or low blood flow regardless of the cerebral perfusion pressure, but impaired cerebral autoregulation may lead to negative results in various neurological conditions such as a traumatic brain injury, intracranial hemorrhage and cerebral infarction.

One of the cerebrovascular diseases related to the cerebral autoregulation is a moyamoya disease. The moyamoya disease refers to a syndrome in which stenosis or occlusion is observed at an end portion of an internal carotid artery in the skull, that is, a beginning portion of an anterior cerebral artery and a middle cerebral artery without any special reason, and abnormal blood vessels called moyamoya vessels are observed near the portion. An occurrence of cerebral infarction in a patient with the moyamoya disease is a major form of neurological damage which is closely related to the impaired cerebral autoregulation.

Accordingly, in order to predict and prevent postoperative complications in the patient suffering from the moyamoya disease, the development of a technique capable of evaluating cerebral autoregulation in real time during surgery is required.

### SUMMARY

It is an object of the present invention to provide an apparatus and method for evaluating cerebral autoregulation in real time during surgery.

To achieve the above object, according to an aspect of the present invention, there is provided an apparatus for evaluating cerebral autoregulation including: a data acquisition unit configured to acquire blood pressure data and oxygen saturation data of a patient undergoing surgery: a correlation coefficient calculation unit configured to calculate correlation coefficients between the acquired blood pressure data and the acquired oxygen saturation data: a filtering unit configured to filter the calculated correlation coefficients using a moving average filter having a predetermined time window; and a cerebral autoregulation evaluation unit configured to evaluate the cerebral autoregulation of the patient undergoing surgery based on the filtered correlation coefficient.

The predetermined time window may be 25 minutes or more and 30 minutes or less.

The correlation coefficient calculation unit may calculate the correlation coefficients between the blood pressure data and the oxygen saturation data for a first time period at a second time interval.

The first time period may be 5 minutes, and the second time interval may be 10 seconds.

The cerebral autoregulation evaluation unit may evaluate the cerebral autoregulation of the patient undergoing surgery as normal if an absolute value of the filtered correlation coefficient is in a first section less than a predetermined threshold value, and evaluate the cerebral autoregulation of the patient undergoing surgery as abnormal if the absolute value of the filtered correlation coefficient is in a second section greater than or equal to the predetermined threshold value.

The apparatus may further include an alarm unit configured to output an alarm based on a cerebral autoregulation evaluation result.

According to another aspect of the present invention, there is provided a method for evaluating cerebral autoregulation including: acquiring blood pressure data and oxygen saturation data of a patient undergoing surgery: calculating correlation coefficients between the acquired blood pressure data and the acquired oxygen saturation data: filtering the calculated correlation coefficients using a moving average filter having a predetermined time window; and evaluating the cerebral autoregulation of the patient undergoing surgery based on the filtered correlation coefficient.

The predetermined time window may be 25 minutes or more and 30 minutes or less.

The step of calculating correlation coefficients may calculate the correlation coefficients between the blood pressure data and the oxygen saturation data for a first time period at a second time interval.

The first time period may be 5 minutes, and the second time interval may be 10 seconds.

The step of evaluating the cerebral autoregulation may evaluate the cerebral autoregulation of the patient undergoing surgery as normal if an absolute value of the filtered correlation coefficient is in a first section less than a predetermined threshold value, and evaluate the cerebral autoregulation of the patient undergoing surgery as abnormal if the absolute value of the filtered correlation coefficient is in a second section greater than or equal to the predetermined threshold value.

The method may further include outputting an alarm based on a cerebral autoregulation evaluation result.

The cerebral autoregulation of a patient undergoing surgery may be evaluated in real time during the surgery. Through this, it is possible to predict side effects that may occur after the surgery in advance and help medical staffs make decisions so as to take appropriate measures during the surgery, thereby preventing the side effects that may occur after the surgery.

### BRIEF DESCRIPTION OF THE DRAWINGS

FIG. 1 is a block diagram illustrating an apparatus for evaluating cerebral autoregulation (“cerebral autoregulation evaluation apparatus”) according to an exemplary embodiment.

FIG. 2 is a block diagram illustrating a computing environment including a computing device suitable for use in exemplary embodiments.

FIG. 3 is a flowchart illustrating a method for evaluating cerebral autoregulation according to an exemplary embodiment.

FIG. 4 is a graph illustrating results where a level to which a cerebral infarction occurrence group could be distinguished was evaluated while changing a time window of a moving average filter.

### DETAILED DESCRIPTION

Hereinafter, embodiments will be described in detail with reference to the accompanying drawings. In denoting reference numerals to components of respective drawings, it should be noted that the same components will be denoted by the same reference numerals although they are illustrated in different drawings. Further, in description of preferred embodiments of the present invention, the publicly known functions and configurations related to the present invention, which are verified to be able to make the purport of the present invention unnecessarily obscure will not be described in detail.

Meanwhile, in respective steps, each of the steps may occur differently from the specified order unless a specific order is clearly described in the context. That is, each of the steps may be performed in the same order as the specified order, may be performed substantially simultaneously, or may be performed in the reverse order.

Further, wordings to be described below are defined in consideration of the functions in the present invention, and may differ depending on the intentions of a user or an operator or custom. Accordingly, such wordings should be defined on the basis of the contents of the overall specification.

It will be understood that, although the terms first, second, etc. may be used herein to describe various components, but these components should not be limited by these terms. These terms are used only to distinguish one component from other components. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

In addition, a division of the configuration units in the present disclosure is intended for ease of description and divided only by the main function set for each configuration unit. That is, two or more of the configuration units to be described below may be combined into a single configuration unit or formed by two or more of divisions by function into more than a single configuration unit. Further, each of the configuration units to be described below may additionally perform a part or all of the functions among functions set for other configuration units other than being responsible for the main function, and a part of the functions among the main functions set for each of the configuration units may be exclusively taken and certainly performed by other configuration units. Each of the configuration units to be described below may be implemented as hardware or software, or may be implemented as a combination of hardware and software.

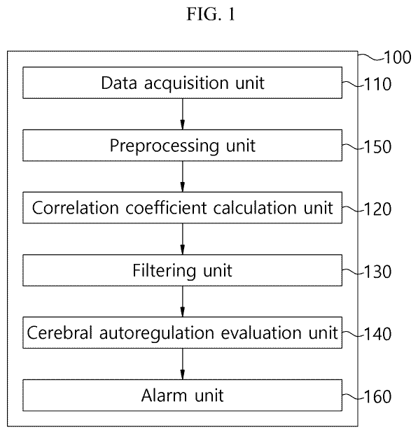

FIG. 1 is a block diagram illustrating a cerebral autoregulation evaluation apparatus according to an exemplary embodiment.

A cerebral autoregulation evaluation apparatus 100 according to an exemplary embodiment is an apparatus capable of evaluating the cerebral autoregulation of a patient undergoing surgery in real time based on blood pressure data and oxygen saturation data of the patient during the surgery, and may be equipped in an electronic device or implemented as a separate apparatus. Here, the electronic device may include a cart type device and a portable device, wherein the portable device may include a personal computer, a laptop computer, a tablet PC, etc., but it is not limited thereto.

Referring to FIG. 1, the cerebral autoregulation evaluation apparatus 100 according to an exemplary embodiment may include a data acquisition unit 110, a correlation coefficient calculation unit 120, a filtering unit 130, and a cerebral autoregulation evaluation unit 140.

The data acquisition unit 110 may acquire the blood pressure data and the oxygen saturation data of the patient undergoing surgery. At this time, the blood pressure data and the oxygen saturation data may be time series data.

For example, the data acquisition unit 110 includes a blood pressure measurement device and an oxygen saturation measurement device, and may acquire the blood pressure data and the oxygen saturation data of the patient undergoing surgery, by measuring a blood pressure and an oxygen saturation of the patient undergoing surgery using the blood pressure measurement device and the oxygen saturation measurement device. At this time, the blood pressure measurement device may be a device which measures the blood pressure using an invasive method, and the oxygen saturation measurement device may be a device which measures the oxygen saturation using near-infrared spectroscopy, but these are only one embodiment, and they are not limited thereto.

As another example, the data acquisition unit 110 may acquire the blood pressure data and the oxygen saturation data of the patient undergoing surgery by receiving blood pressure data and oxygen saturation data of the patient undergoing surgery from an external device which measures and/or stores the blood pressure and/or the oxygen saturation. At this time, the data acquisition unit 110 may use the wired or wireless communication technique. Here, the wireless communication technique may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, ultra-wideband (UWB) communication, Ant+ communication, WIFI communication, Radio Frequency Identification (RFID) communication, 3G communication, 4G communication, 5G communication, or the like, but it is not limited thereto.

The correlation coefficient calculation unit 120 may calculate correlation coefficients between the acquired blood pressure data and oxygen saturation data. At this time, the correlation coefficient may be the Pearson correlation coefficient, but it is not limited thereto. The Pearson correlation coefficient is a numerical value obtained by quantifying a linear correlation between two variables, and may have a value between +1 and −1. Here, +1 may mean a perfect positive linear correlation, 0 may mean no linear correlation, and −1 may mean a perfect negative linear correlation.

For example, the correlation coefficient calculation unit 120 may calculate the correlation coefficients between the blood pressure data and the oxygen saturation data for a first time period at a second time interval. At this time, the first time period may be 5 minutes and the second time interval may be 10 seconds, but these are only an embodiment, and they are not limited thereto.

The correlation coefficient between the blood pressure data and the oxygen saturation data may be referred to as a cerebral oxygenation index (COx).

The filtering unit 130 may filter the correlation coefficients calculated by the correlation coefficient calculation unit 120 using a moving average filter having a predetermined time window. At this time, the predetermined time window may be a value experimentally derived to enable the cerebral autoregulation of the patient undergoing surgery to be evaluated in real time during the surgery. For example, the predetermined time window may be 25 minutes or more, and preferably 25 minutes or more and 30 minutes or less.

The cerebral autoregulation evaluation unit 140 may evaluate the cerebral autoregulation of the patient undergoing surgery based on the correlation coefficients filtered by the moving average filter having the predetermined time window.

For example, as an absolute value of the correlation coefficient filtered by the moving average filter is smaller, the cerebral autoregulation evaluation unit 140 may evaluate that the cerebral autoregulation of the patient undergoing surgery works well.

For another example, the cerebral autoregulation evaluation unit 140 divides the absolute values of the filtered correlation coefficients into a first section less than a predetermined threshold value and a second section greater than or equal to the predetermined threshold value, then may evaluate the cerebral autoregulation of the patient undergoing surgery as normal if the absolute value of the filtered correlation coefficient is in the first section, and evaluate the cerebral autoregulation of the patient undergoing surgery as abnormal (damaged) if the absolute value of the filtered correlation coefficient is in the second section.

According to an exemplary embodiment, the cerebral autoregulation evaluation apparatus 100 may further include a preprocessing unit 150 and/or an alarm unit 160.

The preprocessing unit 150 may preprocess the acquired blood pressure data and oxygen saturation data. For example, the preprocessing unit 150 may remove noise from the acquired blood pressure data and oxygen saturation data. At this time, the preprocessing unit 150 may use the various noise removal techniques known in the art.

The alarm unit 160 may output an alarm based on a cerebral autoregulation evaluation result of the patient undergoing surgery. For example, if it is determined that the cerebral autoregulation evaluation result is abnormal and the abnormal state continues for a predetermined time, the alarm unit 160 may generate and output an alarm. As another example, if a cumulative duration of the abnormal state in the cerebral autoregulation evaluation result is a predetermined time or more, the alarm unit 160 may generate and output an alarm.

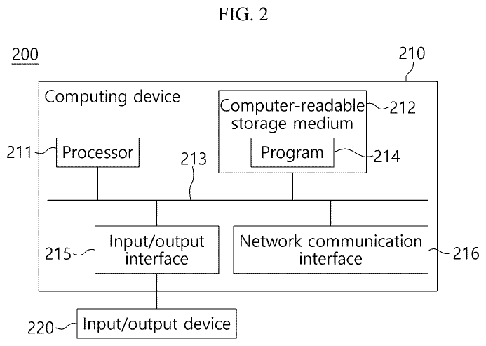

FIG. 2 is a block diagram illustrating a computing environment including a computing device suitable for use in exemplary embodiments. In the illustrated embodiment, the respective components may have different functions and capabilities other than those described below, and may also include additional components other than those described below.

The illustrated computing environment 200 may include a computing device 210. In one embodiment, the computing device 210 may be the cerebral autoregulation evaluation apparatus 100.

The computing device 210 may include at least one processor 211, a computer-readable storage medium 212, and a communication bus 213. The processor 211 may cause the computing device 210 to operate according to the above-described exemplary embodiments. For example, the processor 211 may execute one or more programs stored in the computer-readable storage medium 212. The one or more programs may include one or more computer-executable instructions. When executed by the processor 211, the computer-executable instructions may be configured to cause the computing device 210 to perform operations according to the exemplary embodiments.

The computer-readable storage medium 212 may be configured to store computer-executable instructions or program code, program data, and/or other suitable type of information. A program 214 stored in the computer-readable storage medium 212 may include a set of instructions executable by the processor 211. In one embodiment, the computer-readable storage medium 212 may be a memory (a volatile memory, such as a random access memory, a nonvolatile memory, or a suitable combination thereof), one or more magnetic disk storage devices, optical disk storage devices, flash memory devices, or other types of storage medium accessed by the computing device 210 and capable of storing desired information, or a suitable combination thereof.

The communication bus 213 may connect various other components of the computing device 210 with each other.

The computing device 210 may also include one or more input/output interfaces 215, which provide interfaces for one or more input/output devices 220, and one or more network communication interfaces 216. The input/output interface 215 and the network communication interface 216 may be connected to the communication bus 213. The input/output device 220 may be connected to other components of the computing device 210 through the input/output interface 215. Exemplary input/output devices 220 may include input devices such as a pointing device (such as a mouse or a trackpad), a keyboard, a touch input device (such as a touchpad or a touchscreen), a voice or sound input device, various types of sensor devices and/or photographing devices, and/or output devices such as a display device, a printer, speakers, and/or a network card. Exemplary input/output devices 220 may be included in the computing device 210 as one component which forms the computing device 210, or may be connected to the computing device 210 as a separate device distinct from the computing device 210.

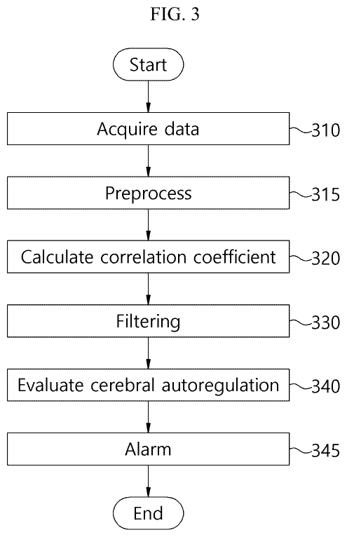

FIG. 3 is a flowchart illustrating a method for evaluating cerebral autoregulation according to an exemplary embodiment. The method for evaluating cerebral autoregulation of FIG. 3 may be performed by the cerebral autoregulation evaluation apparatus 100 of FIG. 1.

Referring to FIG. 3, the cerebral autoregulation evaluation apparatus may obtain blood pressure data and oxygen saturation data of a patient undergoing surgery (310).

For example, the cerebral autoregulation evaluation apparatus includes the blood pressure measurement device and the oxygen saturation measurement device, and may acquire the blood pressure data and the oxygen saturation data of the patient undergoing surgery, by measuring a blood pressure and an oxygen saturation of the patient undergoing surgery using the blood pressure measurement device and the oxygen saturation measurement device.

For another example, the cerebral autoregulation evaluation apparatus may acquire the blood pressure data and the oxygen saturation data of the patient undergoing surgery by receiving blood pressure data and oxygen saturation data of the patient undergoing surgery from an external device which measures and/or stores the blood pressure and/or the oxygen saturation.

The cerebral autoregulation evaluation apparatus may calculate correlation coefficients between the acquired blood pressure data and oxygen saturation data (320). At this time, the correlation coefficient may be the Pearson correlation coefficient, but it is not limited thereto.

For example, the cerebral autoregulation evaluation apparatus may calculate the correlation coefficients between the blood pressure data and the oxygen saturation data for a first time period at a second time interval. At this time, the first time period may be 5 minutes and the second time interval may be 10 seconds, but these are only an embodiment, and they are not limited thereto.

The cerebral autoregulation evaluation apparatus may filter the correlation coefficients using the moving average filter having a predetermined time window (330). At this time, the predetermined time window may be experimentally derived to enable the cerebral autoregulation of the patient undergoing surgery to be evaluated in real time during the surgery, and may be 25 to 30 minutes.

The cerebral autoregulation evaluation apparatus may evaluate the cerebral autoregulation of the patient undergoing surgery based on the correlation coefficients filtered by the moving average filter having the predetermined time window (340).

For example, as an absolute value of the correlation coefficient filtered by the moving average filter is smaller, the cerebral autoregulation evaluation apparatus may evaluate that the cerebral autoregulation of the patient undergoing surgery works well.

For another example, the cerebral autoregulation evaluation apparatus divides the absolute values of the filtered correlation coefficients into a first section less than a predetermined threshold value and a second section greater than or equal to the predetermined threshold value, then may evaluate the cerebral autoregulation of the patient undergoing surgery as normal if the absolute value of the filtered correlation coefficient is in the first section, and evaluate the cerebral autoregulation of the patient undergoing surgery as abnormal if the absolute value of the filtered correlation coefficient is in the second section.

According to an exemplary embodiment, the cerebral autoregulation evaluation apparatus may preprocess the acquired blood pressure data and oxygen saturation data (315). For example, the cerebral autoregulation evaluation apparatus may remove noise from the blood pressure data and the oxygen saturation data acquired in step 310 using the various noise removal techniques.

According to an exemplary embodiment, the cerebral autoregulation evaluation apparatus may output an alarm based on the cerebral autoregulation evaluation result of the patient undergoing surgery (345). For example, if it is determined that the cerebral autoregulation evaluation result is abnormal and the abnormal state continues for a predetermined time, the cerebral autoregulation evaluation apparatus may generate and output an alarm. For another example, if the cumulative duration of the abnormal state in the cerebral autoregulation evaluation result is a predetermined time or more, the cerebral autoregulation evaluation apparatus may generate and output an alarm.

### Experimental Example

In order to evaluate cerebral autoregulation, surgical signals of patients with the moyamoya disease, which is a representative cerebrovascular disease, were collected. The patients were divided into two groups according to whether the cerebral infarction occurs after surgery, and an experiment to evaluate how the cerebral autoregulation differed between the two groups was performed. 10 cases out of a total of 68 surgical records were classified into a cerebral infarction occurrence group.

An average of the correlation coefficients between the blood pressure and the oxygen saturation collected during an entire surgery time was 0.78 level of area under the receiver operating characteristic curve (AUROC) value, which significantly classified the two groups. However, when the correlation coefficient was evaluated in real time, a significant difference between the two groups was not found. Therefore, it was intended to apply a moving average filter having different time windows thereto, thus to find a difference between the groups.

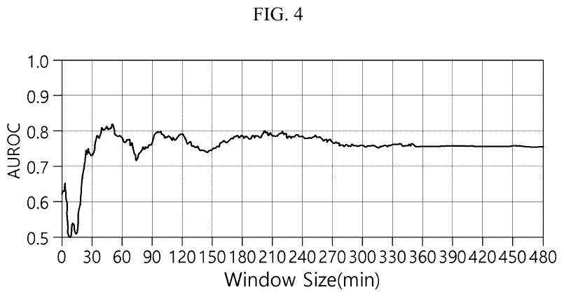

As a result of evaluating the level to which the cerebral infarction occurrence group could be distinguished while changing the time window of the moving average filter, the results shown in FIG. 4 might be acquired.

Referring to FIG. 4, it can be seen that the AUROC to predict the cerebral infarction is about 0.74 when the time window size of the moving average filter is 25 minutes, the AUROC to predict the cerebral infarction is about 0.75, and the time window size is 30 minutes. In addition, it can be seen that the AUROC to predict the cerebral infarction has a value between about 0.72 and about 0.82 when the time window size is 30 minutes or more and 300 minutes or less, and it is maintained at about 0.77 when the time window size is 300 minutes or more.

That is, it could be confirmed that, when the time window size of the moving average filter is set to 25 minutes or more, the cerebral infarction occurrence group after surgery could be distinguished at a relatively high level. Through this, it could be confirmed that, when the time window size is set to 25 minutes or more, and preferably 25 minutes or more and 30 minutes or less, the cerebral autoregulation during the surgery might be evaluated in real time at a relatively high level.

The present invention has been described with reference to the preferred embodiments above, and it will be understood by those skilled in the art that various modifications may be made within the scope without departing from essential characteristics of the present invention. Accordingly, it should be interpreted that the scope of the present invention is not limited to the above-described embodiments, and other various embodiments within the scope equivalent to those described in the claims are included within the present invention.

1. An apparatus for evaluating cerebral autoregulation comprising:
a data acquisition unit configured to acquire blood pressure data and oxygen saturation data of a patient undergoing surgery;
a correlation coefficient calculation unit configured to calculate correlation coefficients between the acquired blood pressure data and the acquired oxygen saturation data;
a filtering unit configured to filter the calculated correlation coefficients using a moving average filter having a predetermined time window; and
a cerebral autoregulation evaluation unit configured to evaluate the cerebral autoregulation of the patient undergoing surgery based on the filtered correlation coefficient.

2. The apparatus for evaluating cerebral autoregulation according to claim 1, wherein the predetermined time window is 25 minutes or more and 30 minutes or less.

3. The apparatus for evaluating cerebral autoregulation according to claim 1, wherein the correlation coefficient calculation unit calculates the correlation coefficients between the blood pressure data and the oxygen saturation data for a first time period at a second time interval.

4. The apparatus for evaluating cerebral autoregulation according to claim 3, wherein the first time period is 5 minutes, and the second time interval is 10 seconds.

5. The apparatus for evaluating cerebral autoregulation according to claim 1, wherein the cerebral autoregulation evaluation unit evaluates the cerebral autoregulation of the patient undergoing surgery as normal if an absolute value of the filtered correlation coefficient is in a first section less than a predetermined threshold value, and evaluates the cerebral autoregulation of the patient undergoing surgery as abnormal if the absolute value of the filtered correlation coefficient is in a second section greater than or equal to the predetermined threshold value.

6. The apparatus for evaluating cerebral autoregulation according to claim 1, further comprising an alarm unit configured to output an alarm based on a cerebral autoregulation evaluation result.

7. A method for evaluating cerebral autoregulation, the method comprising:
acquiring blood pressure data and oxygen saturation data of a patient undergoing surgery;
calculating correlation coefficients between the acquired blood pressure data and the acquired oxygen saturation data;
filtering the calculated correlation coefficients using a moving average filter having a predetermined time window; and
evaluating the cerebral autoregulation of the patient undergoing surgery based on the filtered correlation coefficient.

8. The method for evaluating cerebral autoregulation according to claim 7, wherein the predetermined time window is in a range of 25 minutes to 30 minutes.

9. The method for evaluating cerebral autoregulation according to claim 7, wherein the calculating of the correlation coefficients comprises calculating the correlation coefficients between the blood pressure data and the oxygen saturation data for a first time period at a second time interval.

10. The method for evaluating cerebral autoregulation according to claim 9, wherein the first time period is 5 minutes, and the second time interval is 10 seconds.

11. The method for evaluating cerebral autoregulation according to claim 7, wherein the evaluating of the cerebral autoregulation comprises evaluating the cerebral autoregulation of the patient undergoing surgery as normal if an absolute value of the filtered correlation coefficient is in a first section less than a predetermined threshold value, and evaluating the cerebral autoregulation of the patient undergoing surgery as abnormal if the absolute value of the filtered correlation coefficient is in a second section greater than or equal to the predetermined threshold value.

12. The method for evaluating cerebral autoregulation according to claim 7, further comprising outputting an alarm based on a cerebral autoregulation evaluation result.
